(19) United States
(12) Patent Application Publication
(10) Pub. No.: US 2010/0216198 A1
(43) Pub. Date: Aug. 26, 2010

(54) METHOD FOR THE SYNTHESIS OF OMEGA-UNSATURATED FATTY ACIDS

(75) Inventor: Jean-Luc Dubois, Millery (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkerna France, Colombes (FR)

(21) Appl. No.: 12/678,622

(22) PCT Filed: Sep. 17, 2008
(86) PCT No.: PCT/FR2008/051665

§ 371 Date: Mar. 17, 2010

(30) Foreign Application Priority Data
Sep. 20, 2007 (FR) 0757694

(51) Int. Cl. C12P 7/64

(52) U.S. Cl. 435/134

(57) ABSTRACT

The invention relates to a method for the synthesis of short-chain ω-unsaturated fatty acids of general formula CH₂═CH—(CH₂)ₙ—COOR in which n is an integer between 2 and 11 and R is H or an alkyl radical containing from 1 to 4 carbon atoms, from long-chain monounsaturated fatty acids or esters of formula CH₃—(CH₂)ₘ—CH═CH—(CH₂)ₚ—COOR in which m and p are integers, either identical or different, between 2 and 11, such that the molecule contains at least 10 adjacent carbon atoms in the main chain, said method comprises the first step of submitting the long-chain monounsaturated fatty acid charge to an oxidation by fermentation using a microorganism containing enzymes of oxygenase type, thus leading to the formation of α,ω-diacids, and then a second step of subjecting the product from the first step to a catalytic metsthesis crossed with ethylene for forming two short-chain ω-unsaturated fatty acids having a main chain length relative to that of the starting unsaturated fatty acid or ester of between 0.2 and 0.8, and preferably 0.35 and 0.75.

The invention relates to a method for the synthesis, by metathesis, of ω-unsaturated fatty acids or esters from a natural monounsaturated fatty acid or fatty ester.

ω-Unsaturated acids or esters are molecules that are useful in an entire series of reactions, such as carbonylation, hydroformylation and epoxydation in particular. These acids are not generally prepared according to the usual methods for the chemical synthesis of unsaturated acids, which generally result in α- or β-unsaturated acids and not in ω-unsaturated fatty acids. Consequently, in practice, acids of this type originate from the conversion of more complex unsaturated acids and, in particular, fatty acids of natural origin, which are subjected to more or less complex treatments.

Thus, an industrial process for the manufacture of 11-aminoundecanoic acid is known which uses, as starting material, ricinoleic acid extracted from castor oil, which is the basis for the synthesis of Rilsan®. In this process, the ω-undecylenic acid, in its ester form, undecylenate, is synthesized as an intermediate product by pyrolysis of the charge which is accompanied by the formation of heptanaldehyde as coproduct. This process is described in the work “Les Precédés de Pétrochimie” [Petrochemical Processes] by A. Chauvel et al. published in Editions TECHNIP (1986).

Another method for the synthesis of “short-chain” ω-unsaturated fatty acids or esters from “long-chain” fatty acids or esters consists in carrying out a cross metathesis of these long-chain acids with ethylene, an operation known as ethenolysis. This method is mentioned in particular in the article by Soon Hyeok Hong et al. “Prevention of Undesirable Isomerization during Olefin Metathesis” published in the Journal of the American Chemical Society 2005, 127, 17160-17161, which mentions, on this occasion, the risk of competition between isomerization and metathesis. It is also cited in the article by J. C. Mol “Catalytic metathesis of unsaturated fatty acid esters and oils” published in Topics in Catalysis Vol. 17, Nos 1-4, February 2004 pages 97 to 104.

Formation of the “short-chain” ω-unsaturated fatty acid always accompanied by concomitant formation of an α-olefin should be observed in the methods thus described. This has the drawback, in terms of the cost of the process, of having to find, on site, an industrial use for this α-olefin which is a by-product of the reaction. In fact, since the market price for α-olefins is about that of monounsaturated fatty acids, the ω-unsaturated fatty acid part alone must, from the financial point of view, bear the entire cost of synthesis.

This reaction is illustrated by the scheme below illustrating the ethenolysis of petroselinic acid.

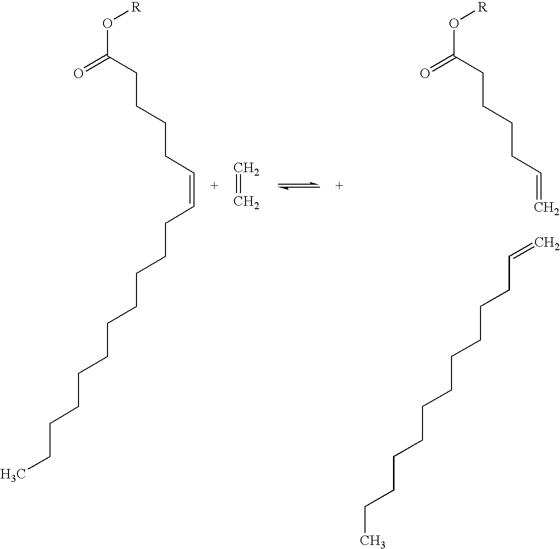

The invention aims to overcome this drawback by proposing a method which produces only “short-chain” ω-unsaturated fatty acids from fatty acids or esters of natural origin.

In the method of the invention, the fatty acid can be treated either in its acid form or in its ester form. The change from one form to the other is carried out by methanolysis, esterification or hydrolysis. In the interests of facility of the disclosure, the subsequent description will be mainly directed toward the acid, in the knowledge that the reaction is immediately transposable to the ester.

The subject of the present invention is a method for the synthesis of “short-chain” ω-unsaturated fatty acids or esters of general formula CH₂═CH—(CH₂)ₙ—COOR in which n is an integer between 2 and 11 and R is H or an alkyl radical containing from 1 to 4 carbon atoms, from long-chain monounsaturated fatty acids or esters of natural origin and of formula CH₃—(CH₂)ₘ—CH═CH—(CH₂)ₚ—COOR in which m and p are integers, which may be identical or different, between 2 and 11, such that the molecule contains at least 10 adjacent carbon atoms in the main chain, said method comprising a first step of subjecting the long-chain mainly monounsaturated fatty acid or ester charge to an oxidation by fermentation leading to the formation of long-chain α-ω-diacids, and then a second step of subjecting the product resulting from the first step to cross-metathesis with ethylene so as to produce the formation of two “short-chain” ω-unsaturated fatty acids or esters.

In the method of the invention, the expression “short-chain” ω-unsaturated fatty acids or esters is intended to mean compounds generally containing a number of adjacent carbon atoms of between 5 and 14, this number always being less than the number of carbon atoms of the main chain of the monounsaturated fatty acid or ester used for its synthesis. The chain length of the omega-unsaturated acid relative to that of the starting unsaturated fatty acid is between 0.2 and 0.8, and preferably between 0.35 and 0.75, and even more preferably between 0.42 and 0.72, and even more preferably between 0.45 and 0.64. In one preferred variant of the method of the invention, these omega-unsaturated acids or esters will contain from 6 to 14 carbon atoms per molecule.

In the method of the invention, the expression “mainly monounsaturated fatty acid or ester charge” is intended to mean a charge having a content, by weight, of monounsaturated fatty acids or esters of greater than 50%, preferably greater than 80%.

According to the charge used and the conditions for the oxidation by fermentation, either two different “short-chain” ω-unsaturated fatty acids are obtained as final product or, if the diacid formed is symmetrical, a single “short-chain” ω-unsaturated fatty acid is obtained as final product, which is particularly advantageous in terms of the yield of the method.

In the method of the invention, the expression “long-chain monounsaturated fatty acids or esters of natural origin” is intended to mean an acid or ester derived from plant or animal environments, including algae, more generally from the plant kingdom, and therefore renewable, containing at least 10 and preferably at least 14 carbon atoms per molecule.

By way of examples of such acids, mention may be made of C10 acids, obtusilic (cis-4-decenoic) acids, C12 acids, lauroleic (cis-5-dodecenoic) and linderic (cis-4-dodecenoic) acids, C14 acids, myristoleic (cis-9-tetradecenoic), physeteric (cis-5-tetradecenoic) and tsuzuic (cis-4-tetradecenoic) acids, C16 acid, palmitoleic (cis-9-hexadecenoic) acid, C18 acids, oleic (cis-9-octadecenoic) acid, elaidic (trans-9-octadecenoic), petroselinic (cis-6-octadecenoic), vaccenic (cis-11-octadecenoic) and ricinoleic (12-hydroxy-cis-9-octadecenoic) acids, C20 acids, gadoleic (cis-9-eicosenoic) and gondoic (cis-1′-eicosenoic) acids, cis-5-eicosenoic acid and lesquerolic (14-hydroxy-cis-11-eicosenoic) acid, and C22 acids, cetoleic (cis-1′-docosenoic) and erucic (cis-13-docosenoic) acids.

The first step is carried out by fermentation using a microorganism, such as a bacterium, a fungus or a yeast, enabling oxidation of the fatty acid or ester of the charge. Microorganisms containing enzymes of oxygenase type, capable of oxidizing the charge by forming a trivalent function of acid —COOH or ester —COOR, will preferably be used.

This fermentation may, for example, be carried out in the presence of a strain of Candida tropicalis containing Cytochrome P450 Monooxygenase enzymes such as those described in the publication by W. H. Eschenfeldt at al. “Transformation of fatty Acids Catalyzed by Cytochrome P450 Monooxygenase Enzymes of Candida tropicalis” published in Applied and Environmental Microbiology, October 2003 p. 5992-5999 and patents FR 2,445,374, U.S. Pat. No. 4,474,882, U.S. Pat. No. 3,823,070, U.S. Pat. No. 3,912,586, U.S. Pat. No. 6,660,505, U.S. Pat. Nos. 6,569,670 and 5,254,466.

The metathesis reactions carried out in the second step have been known, but have never been described, with diacids for a long time, even though the industrial applications thereof are relatively limited. Reference may be made, with regard to the use thereof in the conversion of fatty acids (esters), to the article by J. C. Mol “Catalytic metathesis of unsaturated fatty acid esters and oil” published in Topics in Catalysis Vol. 27, Nos. 1-4, February 2004 (Plenum Publishing Corporation).

The catalysis of the metathesis reaction has been the subject of a very large number of studies and the development of sophisticated catalytic systems. Mention may, for example, be made of the tungsten complexes developed by Schrock et al., J. Am. Chem. Soc. 108 (1986) 2771, or Basset at al. Angew. Chem., Ed. Engl. 31 (1992) 628. More recently, “Grubbs'” catalysts (Grubbs et al. Angew. Chem., Ed. Engl. 34 (1995) 2039 and Organic Lett. 1 (1999) 953), which are ruthenium-benzylidene complexes, have appeared. This catalysis is homogeneous catalysis. Heterogeneous catalysts based on metals such as rhenium, molybdenum and tungsten, deposited on alumina or silica, have also been developed. Finally, studies have been carried out for the preparation of immobilized catalysts, i.e. of catalysts for which the active ingredient is that of the homogeneous catalyst, in particular ruthenium-carbene complexes, but which is immobilized on an inactive support. The objective of these studies is to increase the selectivity of the reaction with respect to parasitic reactions such as “homometatheses” between the reactants brought together. They relate not only to the to structure of the catalysts but also to the effect of the reaction medium and the additives that can be introduced.

In the method of the invention, any active and selective metathesis catalyst may be used. Ruthenium-based and rhenium-based catalysts will, however, preferably be used.

The second step is illustrated by examples of short-chain fatty diacid synthesis hereinafter. All the mechanisms described in detail below illustrate, in order to facilitate the disclosure, the acid form. However, the metathesis is as effective with an ester and often is even more effective. In the same way, the schemes illustrate reactions with the cis-isomer of the acids (or esters); the mechanisms are just as applicable to the trans-isomers.

The reaction process of this second step using oleic diacid, or α-ω-9-octadecenedioic acid, and ethylene is the following:

HOOC—(CH₂)₇—CH═CH—(CH₂)₇—COOH+CH₂═CH₂ [symbol] 2CH₂═CH—(CH₂)₇—COOH.

It can be seen that the method has, in this case, a most particular advantage in so far as the formation of a coproduct is avoided, so as to produce only 9-decenoic acid.

The reaction process is illustrated by scheme 1 below.

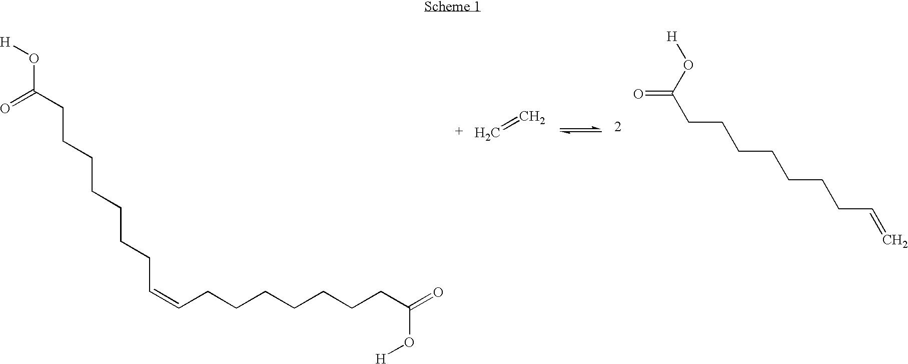

Under certain fermentation conditions, macadamia oil or sea buckthorn oil is partially oxidized so as to give a C16 diacid, which is α,ω-7-hexadecenedioic acid, from the palmitoleic acid contained in these oils.

The reaction process of this second step using this diacid and ethylene so as to produce two different ω-unsaturated acids, which are 9-decenoic acid and 7-octenoic acid, can be described by means of the following mechanism:

HOOC—(CH₂)₅—CH═CH—(CH₂)₇—COOH+CH₂═CH₂ [symbol] CH₂═CH—(CH₂)₇—COOH+CH₂═CH—(CH₂)₅—COOH.

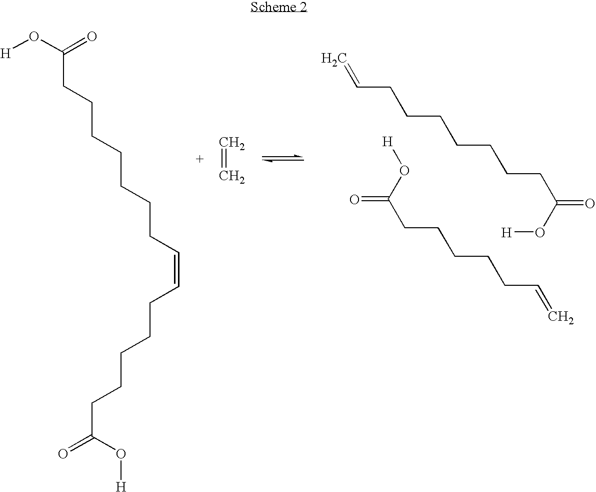

Lauroleic acid is converted, during the fermentation, to α,ω-5-dodecenedioic acid. The second step of the method will produce two ω-olefinic acids of different lengths, which are 7-octenoic acid and 5-hexenoic acid, according to the following process:

HOOC—(CH₂)₅—CH═CH—(CH₂)₃—COOH+CH₂═CH₂ [symbol] CH═CH₂—(CH₂)₅—COOH+CH₂═CH—(CH₂)₃—COOH.

The myristoleic acid is converted, during the fermentation, to α,ω-5-tetradecenedioic acid. The second step of the method will produce two ω-olefinic acids of different lengths, which are 9-decenoic acid and 5-hexenoic acid, according to the following process:

HOOC—(CH₂)₇—CH═CH—(CH₂)₃—COOH+CH₂═CH₂ [symbol] CH₂═CH—(CH₂)₇—COOH+CH₂═CH—(CH₂)₃—COOH.

The invention is illustrated by the examples hereinafter.

### EXAMPLES

### Example 1

### Oleic Acid Fermentation

In this example, a yeast containing at least one oxygenase enzyme will be used.

The yeast will be cultured at pH=7, in a medium of deionized water containing sorbitol, trace elements, urea and oleic acid. The mixture will then be sterilized at 120° C. for 15 minutes. A yeast strain will then be inoculated into the culture medium. The culture will be maintained at 30° C. A solution of sodium hydroxide will be added continuously so as to maintain the medium at a pH of 7.0 to 7.5. After culturing for 48 hours, the unsaturated diacid will be recovered by extraction with diethyl ether. After elimination of the solvent by evaporation, crystals will be recovered which, after recrystallization, will have a melting point of 69° C., i.e. equivalent to that described for 9-octadecenedioic acid.

### Example 2

### Erucic Acid Fermentation

Example 1 will be reproduced using erucic acid. 9-docosenedioic acid will be obtained.

### Example 3

### Cross-Metathesis of 9-Octadecenedioic Acid

This example illustrates the synthesis of 9-decenoic acid from 9-octadecenedioic acid. This second step of the method will be a cross-metathesis with ethylene. A complex catalyst of the bis(pyridine)ruthenium type such as that described in the publication by Chen-Xi Bai et al., Org. Biomol. Chem., (2005), 3, 4139-4142, will be used for this reaction. The reaction will be carried out in CH₂Cl₂, at a molar concentration of ethylene which is twice that of the 9-octadecenedioic acid, at a temperature of 80° C., and for 12 hours, in the presence of the catalyst at a concentration of 1 mol % relative to the 9-octadecenedioic acid. The yields will be determined by chromatographic analysis and the yield of 9-decenoic acid CH₂═CH—(CH₂)₇—COOH will be substantially equimolar.

### Example 4

### Cross-Metathesis of 9-Docosenedioic Acid

Example 3 will be reproduced with the diacid of example 2: 9-docosenedioic acid. A substantially equimolar yield of 9-deceneoic acid and 13-tetradeceneoic acid will be obtained.

### Example 5

### Ethenolysis of the Unsaturated C18 Diester

1 g of methyl 9-octadecenedioate (3 mmol), 47 mg (7.5×10⁻² mmol) of second-generation Hoveyda-Grubbs catalyst (1,3-bis(2,4,6-trimethylphenyl)-2-imidazolidinylidene)dichloro(o-isopropoxyphenylmethylene)ruthenium), available from the company Aldrich, and 15 ml of toluene distilled over sodium benzophenone are charged to a 50 ml Schlenk tube purged with nitrogen. 170 mg of ethylene (6 mmol) are added and the mixture is left to react for 3 hours at 20° C. with magnetic stirring. The reaction mixture is analyzed by gas chromatography. The conversion of the unsaturated diester is 83%. The yield in terms of methyl 9-decenoate is 64%.

1) A method for the synthesis of short-chain ω-unsaturated fatty acids or esters of general formula CH₂═CH—(CH₂)ₙ—COOR in which n is an integer between 2 and 11 and R is H or an alkyl radical containing from 1 to 4 carbon atoms, from long-chain monounsaturated fatty acids or esters of formula CH₃—(CH₂)ₘ—CH═CH—(CH₂)ₚ—COOR in which m and p are integers between 2 and 11, such that the molecule contains at least 10 adjacent carbon atoms in the main chain, said method comprising a first step of subjecting the long-chain monounsaturated fatty acid or ester to an oxidation by fermentation using a microorganism containing enzymes of oxygenase type, leading to the formation of long-chain α,ω-diacids or -diesters, and then a second step of subjecting the product resulting from the first step to catalytic cross-metathesis with ethylene in the presence of ruthenium- and rhenium-based catalysts so as to produce the formation of two short-chain ω-unsaturated fatty acids or esters having a main chain length, relative to that of the starting unsaturated fatty acid or ester, of between 0.2 and 0.8, and preferably 0.35 and 0.75.

2) The method as claimed in claim 1, characterized in that the first step is carried out in the presence of a strain of Candida tropicalis containing Cytochrome P450 Monooxygenase enzymes.

3) The method as claimed in claim 1, characterized in that 9-decenoic acid is synthesized from oleic acid.

4) The method as claimed in claim 1, characterized in that 9-decenoic acid and 13-tetradeceneoic acid are synthesized from erucic acid.

5) The method as claimed in claim 1 characterized in that said microorganism containing enzymes of oxygenase type is selected from the group consisting of a bacterium, a fungus and a yeast.
